(19) United States
(12) Patent Application Publication
(10) Pub. No.: US 2024/0394166 A1
(43) Pub. Date: Nov. 28, 2024

(54) METHOD FOR OPERATING A DIALYSIS SYSTEM

(71) Applicant: B. Braun Avitum AG, Melsungen (DE)

(72) Inventors: Stephan Krietemeyer, Melsungen (DE); Philipp Odernheimer, Melsungen (DE); Nino Lieb, Melsungen (DE); Jonas Giesa, Melsungen (DE)

(21) Appl. No.: 18/671,067

(22) Filed: May 22, 2024

(30) Foreign Application Priority Data
May 24, 2023 (DE) 10 2023 113 670.4

(51) Int. Cl. G06F 11/30; A61M 1/14

(52) U.S. Cl. CPC G06F 11/3058; A61M 1/14

(57) ABSTRACT

A dialysis system includes a plurality of hydraulically connected components and a control device. A method of operating the dialysis system includes receiving component operating data from the components, the component operating data including at least demand data for an upcoming task to be performed by the respective component, determining an expected resource consumption of the component based on the demand data, monitoring of an actual resource consumption of the components, determining a deviation of the actual resource consumption from the expected resource consumption, and detecting a leakage in the dialysis system if the deviation is outside a predetermined range.

### CROSS-REFERENCE TO RELATED APPLICATION

This application claims priority under 35 U.S.C. § 119 to German Application No. 102023 113 670.4, filed on May 24, 2023, the content of which is incorporated by reference herein in its entirety.

### FIELD

Disclosed are a method of operating a dialysis system, a dialysis system, a system comprising a dialysis system, a computer program product and a computer-readable medium.

### BACKGROUND

Until now, various components of dialysis systems, also referred to here as medical devices, have been only hydraulically connected to each other and are programmed individually and usually manually. In such systems, leakages in the overall system cannot be reliably detected.

### SUMMARY

An object underlying the present disclosure is therefore to provide a method for operating a dialysis system that reliably detects leakages.

The present disclosure relates to a method of operating a dialysis system comprising a plurality of hydraulically connected components and a control device. The method comprises receiving, by the control device, component operating data from the components, wherein the component operating data comprises at least demand data for an upcoming task to be performed by the respective component. The method also comprises determining an expected resource consumption of the (respective) component based on the demand data. This may, for example, be the expected resource consumption for the upcoming task to be performed by the component. The method also comprises monitoring, by the control device, of an actual resource consumption of the components, determining, by the control device, a deviation of the actual resource consumption from the expected resource consumption, and detecting, by the control device, a leakage in the dialysis system if the deviation is outside a predetermined range. The (respective) operating data can be received from one or more of the connected components, in particular from all of the connected components.

In this way, a leakage in the dialysis system can be reliably detected. With known systems, this type of leakage detection is not possible, particularly at system level. At best, leakages can be detected on individual components by individually monitoring the respective component, but in some cases, this is difficult or impossible.

The dialysis system can be part of a dialysis center or a dialysis practice, for example.

The components, also referred to as medical devices, can include dialysis machines, hot cleaning devices, reverse osmosis devices, concentrate mixing systems or the like.

The components being hydraulically connected means that fluid can be transported, for example pumped, between the components. The hydraulic connection can take the form of tubes and/or pipes. The hydraulic connection can be direct or indirect. The dialysis system may include valves that can be used, for example, to adjust a flow through the dialysis system, such as automatically controllable valves.

The control device can be integral with one of the components or separate from the components.

The control device can be configured to receive data from the components by means of a data connection, in particular a bi-directional data connection, with the respective component. The data connection can be wireless or wired.

The term component operating data is to be understood broadly and may include, for example, data relating to the operation of a component, such as data representing a current and/or planned operating mode of the component and/or data representing the current and/or planned settings of operating parameters and/or data representing the current state of the component, for example, on-state, off-state, or error state. The component operating data may also include an expected result, such as an expected output.

As seen above, the component operating data comprises at least demand data indicating an expected resource consumption of the (respective) component for an upcoming task to be executed by the component. In particular, the component operating data may indicate the task to be performed and the associated expected resource consumption.

The demand data can indicate an expected resource consumption for one or more resources, for example a water consumption and an energy consumption. The demand data does not have to indicate the expected resource consumption in a specific form of representation, for example specific units. It is sufficient for the demand data to be configured in such a way that the resource consumption can be derived from the demand data, for example by the control device.

The measure of resource consumption can be defined according to the application. For example, the resource consumption can be defined as the difference between the withdrawal quantity and the output quantity, for example the inflowing and outflowing fluid volume or absorbed heat and residual heat, or as the withdrawal quantity, for example electricity consumption.

The method also includes monitoring, by the control device, of an actual resource consumption of the (respective) component.

The actual resource consumption of the component can, for example, be determined using data that the component itself collects. In particular, the component can determine the resource consumption itself using this data.

Alternatively, the actual resource consumption of the (respective) component can be determined by the control device. For this purpose, the component can transmit the collected data to the control device.

Resource consumption can also be determined using measurement data that is not collected by the (respective) component itself but, for example, by sensors that are not part of the (respective) component.

The sensors can be arranged at the (respective) component. For example, such sensors can determine the withdrawal and output quantity of a resource.

At least one sensor that collects data to determine the resource consumption of a component can be located in other areas of the system, i.e. not at the (respective) component.

For example, a volume flow can be measured at various points in the system and does not have to be measured directly at the input or output of a component.

Alternatively or additionally, a component can collect data that is used to determine the actual resource consumption of another component, for example by assuming that the output quantity measured for one component corresponds to the withdrawal quantity of another component.

For example, instead of measuring the inflowing and outflowing volume of a component, only the inflowing volume of components that are hydraulically in direct succession can be measured. As already seen above, the measure of resource consumption can be suitably defined for the application and the measurement can be carried out accordingly.

The monitoring of the actual resource consumption of the (respective) component can in particular comprise a continuous or, for example, at regular intervals, repeated determination of the resource consumption. For example, the resource consumption can be determined after predetermined time intervals, either in the form of a current measured value or integrated measured values, in particular in the form of measured values integrated over the previous time interval.

The method also comprises determining, by the control device, a deviation of the actual resource consumption from the expected resource consumption.

The actual resource consumption can be determined component by component or collectively for several components. A collective determination of resource consumption is described below in connection with an exemplary ring system to which several components are connected.

If the actual resource consumption is determined collectively for several components, it is understood that accordingly the collective resource consumption for these components is to be considered as the expected resource consumption.

Determining the deviation, in particular quantifying the deviation, can, for example, involve determining a difference and/or a quotient of actual and expected resource consumption.

The deviation can be determined, for example, on the basis of the respective deviations between individual values of the actual and expected resource consumption and/or the deviation between one or more time averages of the actual and expected resource consumption and/or on the basis of the deviation between one or more integrated or cumulative values of the actual and expected resource consumption.

The method also includes detection, by the control device, of a leakage in the dialysis system if the deviation is outside a predetermined range.

The range can be regarded as a tolerance range. A certain deviation can, for example, be due to measurement errors or negligible losses. Due to the tolerance range, such a deviation will not immediately lead to a leakage being detected. This reduces false-positive events.

For example, the predetermined range can be defined by one or more predetermined threshold values. The method can include detecting a leakage if a predetermined upper threshold value is exceeded and/or if a predetermined lower threshold value is undershot.

For example, a leakage can be detected if the difference between actual and expected resource consumption exceeds an upper limit.

As another example, a leakage can be recognized if a quotient of expected and actual resource consumption falls below a lower threshold value or a quotient of actual and expected resource consumption exceeds an upper threshold value.

A leakage can be detected directly, for example by balancing volume flows. However, in many cases a leakage will also influence the consumption of other resources, so that indirect leakage detection is also possible. For example, an excessively high withdrawal of electricity may indicate that a pressure drop in the system requires a higher pump power. The pressure drop in turn can indicate a leakage.

To summarize, the control device uses the demand data, in particular the expected resource consumption, and an actual resource consumption to determine whether there is a leakage. The present disclosure enables reliable system-wide monitoring and detection of leakages.

According to the present disclosure, the expected resource consumption can be represented by one or more demand parameters and the actual resource consumption can be represented by one or more consumption parameters, wherein one demand parameter and one consumption parameter form a parameter pair and the deviation is determined for at least one of the parameter pairs.

A parameter pair may, for example, comprise an expected withdrawal quantity and an actual withdrawal quantity of a specific resource, for example fluid volume, heat quantity or electricity. A further parameter pair can, for example, comprise an actual and an expected difference between a withdrawal quantity and an output quantity of a resource, such as a fluid.

According to the present disclosure, a respective deviation of the actual resource consumption from the expected resource consumption can be determined for several, in particular all, parameter pairs and a leakage can be detected if at least a predetermined number of the deviations is outside a predetermined range (predetermined) for the parameter pair.

This means that a leakage may only be determined/detected if there is a deviation for at least the predetermined number of parameter pairs that lies outside the predetermined range (predetermined) for the parameter pair, i.e. is not permissible.

The predetermined range can, for example, be specified in the form of upper and/or lower threshold values, as explained above.

The predetermined number can be greater than or equal to one, in particular greater than or equal to two.

For example, the predetermined number may be one. This means that a leakage may be detected if at least one of the deviations is outside the predetermined range for the parameter pair. This means that a leakage can be detected even if the deviation for just one parameter pair is outside the predetermined range.

This enables particularly reliable detection of all possible leakage events.

Alternatively, the specified number may be greater than one, in particular two or more. In this case, for example, a leakage may only be detected if the respective deviation is outside the predetermined range for at least the predetermined number of parameter pairs, e.g. for at least two parameter pairs.

The occurrence of false-positive leakage detections can be reduced by specifying a number greater than one. For example, a deviation for a parameter pair can be due to a measurement error. If another parameter pair is used, it can be avoided that a false-positive leakage detection occurs due to the measurement error.

According to the present disclosure, the components may comprise one or more consumers and the monitoring of actual resource consumption may comprise the control device receiving data indicative of an actual fluid withdrawal quantity for each of the consumers.

The data on the fluid withdrawal quantity can be determined by the respective consumer or by means of an external sensor. The data can contain the fluid withdrawal quantity or be configured in such a way that the control device can derive the fluid withdrawal quantity from it.

According to the present disclosure, the monitoring of actual resource consumption may comprise a balancing of an inflowing fluid volume of fluid flowing into a ring system and an outflowing fluid volume of fluid flowing out of the ring system. The ring system may, for example, comprise a ring line to which components of the dialysis system are connected.

A balancing can include determining the total inflowing fluid volume, for example integrated or added up over time and/or for different inflows, and the total outflowing fluid volume, for example integrated or added up over time and/or for different outflows. It is also possible to determine the expected difference between the total inflowing fluid volume and the total outflowing fluid volume, for example based on the total expected fluid consumption, determined for example from the demand data.

The balancing can additionally or alternatively be carried out for one or more sections of the ring system.

For example, one or more components, such as the above-mentioned consumers, can be connected to the ring system. Based on the expected resource consumption, here for example the expected fluid consumption, of the components, it is possible to determine how much outflowing fluid volume is to be expected for a certain inflowing fluid volume.

If the actual outflowing fluid volume is less than expected for the inflowing fluid volume, this may indicate a leakage.

The method may include detecting a leakage in the dialysis system if the deviation of the expected and actual outflowing fluid volume is outside a predetermined range. In particular, the expected and actual outflowing fluid volumes may then represent the above-mentioned parameter pair.

An expected outflowing fluid volume of fluid flowing out of the ring system can be determined on the basis of the expected withdrawal quantities of the individual components connected to the ring system.

Balancing, especially if it is only carried out for the entire ring system and not for individual sections, allows a less complex and yet reliable detection of a leakage in the system.

It should be noted here that the detection of a leakage according to the present disclosure refers to the presence of a leakage. Determining where the leakage occurs can, but does not necessarily have to, be part of the method. Therefore, balancing is sufficient to detect a leakage. If the method should also include determining where the leakage occurs, further parameter pairs can be used for this purpose, for example, if the balancing is not sufficient for this purpose.

According to the present disclosure, the method may comprise that the control device receives measurement data from one or more components of the dialysis system on the inflowing fluid volume and the outflowing fluid volume and performs the balancing based thereon.

According to the present disclosure, the method may comprise the control device receiving and evaluating error messages from the components (e.g. components operated in combination). For example, one of the components may be a dialysis machine. The dialysis machine can include a humidity sensor that is installed in the housing of the dialysis machine. During hot cleaning, the dialysis machine can stop with a leakage alarm. However, the amount of moisture escaping may be so small that the central leakage monitoring system may not detect it reliably/accurately. The dialysis machine can stop its hot cleaning process, which can also result in a corresponding reduction in the expected resource consumption.

The measurement data can be determined using flow meters, for example. The measurement data can be recorded by the respective component itself or by means of sensors that do not belong to the component.

According to the present disclosure, monitoring of the actual resource consumption may comprise monitoring of a development over time of the actual resource consumption, and determining a deviation may comprise determining a deviation of the development over time of the actual resource consumption from the development over time of the expected resource consumption.

The resource consumption can have a certain time curve, in particular a non-constant and in particular non-linear time curve. The time curve can, for example, be characteristic of a specific operating mode. This operating mode can, for example, be reported by the components to the control device.

The control device can then use the development over time as an indicator of the presence of a leakage. For example, an unexpected shape of the time curve, in particular unexpected increases in consumption or peaks, can indicate a leakage.

Looking at the time curve can be advantageous, for example, in cases where individual values are not ideal for reliable leakage detection and integrated values also do not have the necessary informative value due to a certain time curve. In such cases, the time curve as such can be a suitable indicator for the presence of a leakage, optionally in combination with other parameters.

According to the present disclosure, the method may comprise receiving, by the control device, data from at least one external information system and detecting the leakage taking into account the data received from the external information system.

The external information system can be considered as an information system that is not part of the dialysis system. For example, it may be a hospital information system (HIS) or an information system that provides component specifications. In particular, the external information system can be located separately from the dialysis system, especially outside a dialysis center. The data can be received via standard data interfaces.

It is possible, for example, to obtain information from a HIS or a component manufacturer information system that is relevant for the operation of the (respective) component.

Taking such data into account can be used to determine the expected resource consumption of certain components, for example in certain operating modes. This enables reliable detection of leakages according to the present disclosure.

According to the present disclosure, the method may comprise an exchange of data, initiated by the control device, between at least two of the components indirectly via the control device.

For example, the control device can cause data that the control device has received from one of the components to be transmitted to another of the components. The exchange of data between two components initiated by the control device can be unidirectional or bidirectional. The exchange of data initiated by the control device can take the form of multicasting the data received by the control device from one of the components to several of the other components. In particular, the data may be component operating data. For example, a switch-on, a switch-over, a switch-off and/or a malfunction of a component can be reported to the control device and, via the control device, indirectly to one or more other components.

According to the present disclosure, the resource consumption may comprise an energy consumption and/or a water consumption and/or a time requirement.

The energy consumption can, for example, be the amount of energy required to complete the task. The energy consumption can include, for example, the consumption of electrical energy and/or energy in the form of heat. Water consumption can be, for example, the amount of water required to complete the task. The water consumption may be the consumption of a certain type of water, for example treated water. The time requirement can be, for example, the time required to complete the task. Other conceivable resources are ingredients to be added to the water, for example electrolytes, or fluids other than water.

According to the present disclosure, at least some of the components can be only hydraulically connected to each other.

In particular, these components can have no direct data connection. This means that these components can only exchange data indirectly via the control device. Direct data exchange between these components may therefore not be possible. This can ensure improved security, and, in addition, it is not necessary to provide compatible interfaces and protocols.

According to the present disclosure, the components may comprise at least one reverse osmosis device and/or at least one concentrate mixing system and/or at least one hot cleaning system and/or at least one dialysis machine.

The reverse osmosis device can be configured for (dialysis) water treatment. The reverse osmosis device can provide treated water as a resource. In addition to time, the resource consumption of the reverse osmosis device can include water consumption, for example of used water, and energy consumption.

The concentrate mixing system can be configured for mixing (dialysis) concentrate. The concentrate mixing system can provide dialysis concentrate as a resource. The resource consumption of the concentrate mixing system may include, in addition to time, a water consumption, an energy consumption, including electrical energy and heat, and additive. Temperature plays a role in how quickly and how well the concentrate is mixed.

The hot cleaning system may be configured for hot cleaning containers and pipes of the dialysis system. The resource consumption of the hot cleaning system may include, in addition to time, water consumption and energy consumption, including electrical energy and heat.

The dialysis machine may be configured to perform dialysis on a patient. The resource consumption of the dialysis machine may include, in addition to time, a consumption of dialysis concentrate, a consumption of water and an energy consumption, including electrical energy and heat. The dialysis machine may provide used water as a resource.

In particular, the method of the present disclosure can be carried out completely automatically, in particular by the control device, unless explicitly stated otherwise.

The present disclosure also provides a dialysis system comprising a plurality of hydraulically connected components and a control device, wherein the control device is connected to each of the components by means of a data connection, wherein the dialysis system is configured to perform the method according to the present disclosure.

In particular, the data connection can be a data connection that enables bi-directional data exchange, i.e. in particular transmission of data from the control device to the (respective) component and from the (respective) component to the control device.

The present disclosure also provides a system comprising the dialysis system.

The system can also comprise an information system external to the dialysis system, for example a hospital information system (HIS) or an information system that provides component specifications. In particular, the external information system may be physically separate from the dialysis system, especially outside a dialysis center.

With regard to the other features and advantages, please refer to the explanations above.

The present disclosure also provides a computer program product comprising instructions that cause the dialysis system of the present disclosure to perform the method steps according to the present disclosure, in particular as described above.

The present disclosure also provides a computer-readable medium on which the computer program product of the present disclosure is stored.

The features and advantages described in connection with the method also apply accordingly to the dialysis system, system, computer program product and computer-readable medium.

### BRIEF DESCRIPTION OF THE DRAWINGS

Further examples and embodiments are explained below with reference to the figures.

FIG. 1 shows a schematic representation of a system according to the present disclosure;

FIG. 2 shows a schematic representation of a method according to the present disclosure;

FIG. 3 shows a schematic representation of a system according to the present disclosure; and

FIG. 4 shows a schematic representation of a system according to the present disclosure.

### DETAILED DESCRIPTION

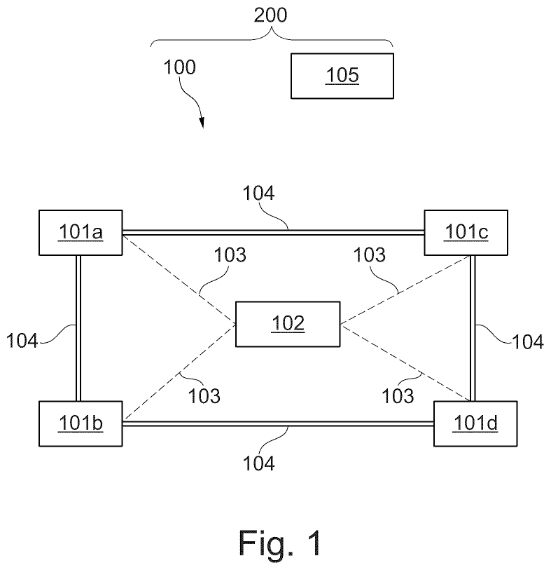

FIG. 1 shows a system 200 according to the present disclosure comprising a dialysis system 100 according to the present disclosure with a plurality of hydraulically connected components 101a, 101b, 101c, 101d and a control device 102, wherein the control device is connected to each of the components by means of a data connection 103, e.g. for bi-directional data exchange. The hydraulic connection is identified by the reference sign 104. In the present example, the components are not connected to each other via a data connection. For example, they can be only hydraulically connected to each other.

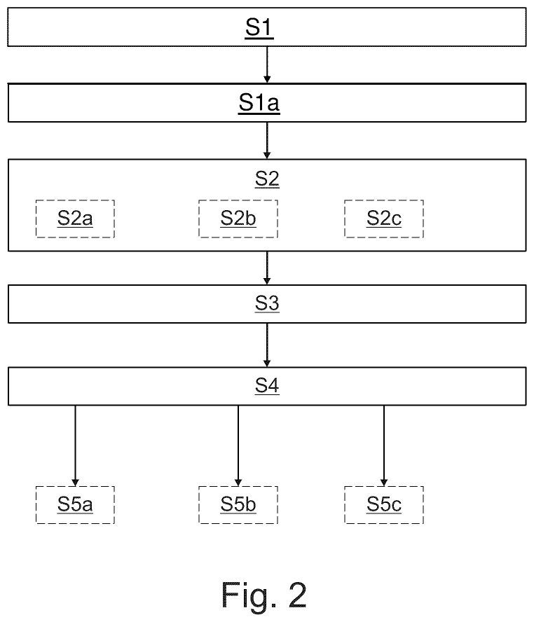

The system shown in FIG. 1 and described above can be configured to carry out the method according to the present disclosure, in particular also the method steps described in connection with FIGS. 2 to 4. In particular, the control device can be configured to carry out or control at least some of the method steps, in particular all method steps.

The system can optionally also comprise an information system 105 external to the dialysis system, for example a hospital information system (HIS) or an information system that provides component specifications. In particular, the external information system may be spatially separated from the dialysis system, in particular outside a dialysis center.

FIG. 2 schematically illustrates a method for operating a dialysis system comprising several hydraulically connected components, for example medical devices, and a control device, according to the present disclosure.

The method comprises, in step S1, receiving component operating data from the components by the control device. The component operating data comprising at least demand data for an upcoming task to be performed by the respective component.

The method comprises, in step S1a, an expected resource consumption of the (respective) component based on the demand data. This is, for example, the expected resource consumption for the upcoming task to be performed by the component. The resource consumption may include, for example, an energy consumption and/or a water consumption and/or a time requirement.

The method further comprises, in step S2, monitoring of an actual resource consumption of the components by the control device.

The monitoring of the actual resource consumption may, for example, comprise monitoring of a development over time of the actual resource consumption, in optional step S2a.

The monitoring of actual resource consumption, when the components comprise one or more consumers, may comprise, in optional step S2b, the control device receiving, for each of the consumers, data indicating an actual resource consumption, for example an actual fluid withdrawal quantity.

The monitoring of the actual resource consumption can also include a balancing of an inflowing fluid volume of fluid flowing into a ring system and an outflowing fluid volume of a fluid flowing out of the ring system, in optional step S2c.

The method further comprises, in step S3, determining a deviation of the actual resource consumption from the expected resource consumption by the control device.

Determining a deviation can include, for example, determining a deviation of the above-mentioned development over time of the actual resource consumption from the development over time of the expected resource consumption.

The method further comprises, in step S4, detecting a leakage in the dialysis system by the control device if the deviation is outside a predetermined range.

The leakage can also be detected, for example, taking into account data received from an external information system. The method can optionally include receiving this data in step S5a.

The method may comprise the control device receiving, in step S5b, measurement data from one or more components of the dialysis system on the inflowing fluid volume and the outflowing fluid volume. This data can be used to detect the leakage. In particular, balancing can be performed based on this data.

The method may comprise, in step S5c, an exchange of data, initiated by the control device, between at least two of the components indirectly via the control device. This data can be used when detecting the leakage.

Further embodiments and advantages of methods or systems according to the present disclosure are described below.

In current methods and systems, operating parameters are typically set on each individual medical device of a dialysis system (for example, a time window is programmed for a planned action) and, if possible and desired, the component is monitored for leakages.

With current methods and systems, it is not possible to monitor leakages throughout the entire system, for example during water withdrawal, as the withdrawal quantities of the consumers are not known.

According to the present disclosure, all hydraulically connected devices can register their requirements, for example for the dialysis-free period, with the central control device.

Components of the dialysis system can, for example, report their respective operating mode to the control device, from which the control device derives the requirements, or provide requirement values directly. The components or other elements of the dialysis system can also report data from which actual requirements are derived to the control device. The control device can then detect leakages if there is more than a predefined deviation between actual and expected requirements.

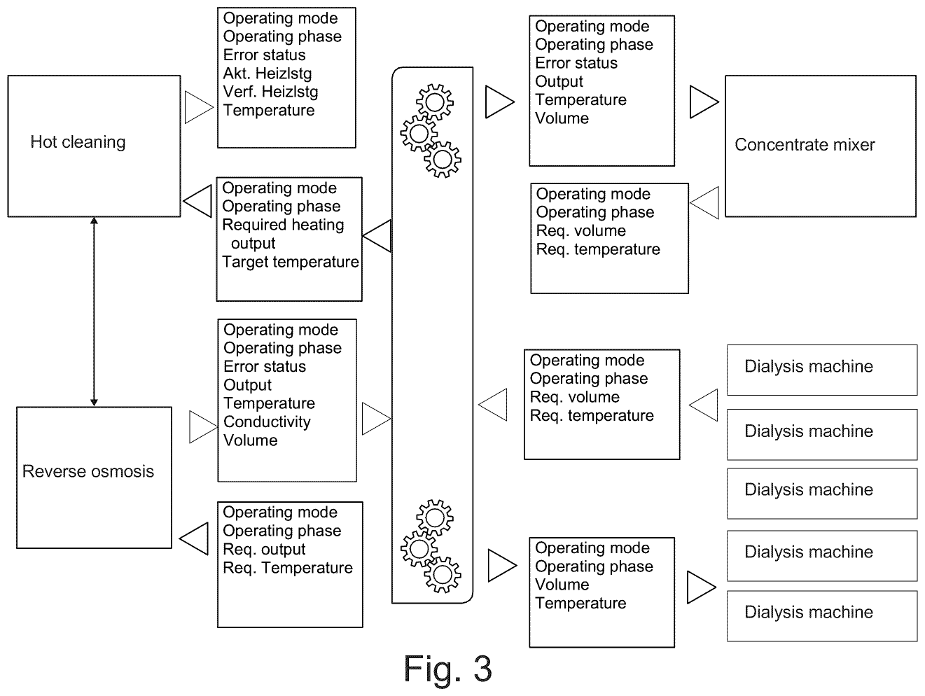

FIG. 3 shows a schematic representation of the possible connected active components/medical devices in a dialysis system according to the present disclosure. The information pointing to the central control device is provided by the connected components/medical devices. The information pointing out from the central control device is provided by the central control device to components.

In particular, the components “hot cleaning”, “reverse osmosis”, “concentrate mixer” and “dialysis machine” are shown here as examples.

The components can each report the operating mode and operating phase to the control unit. In addition, error status, current heating power (e.g. in kW), available heating power (e.g. in kW) and temperature can also be reported by the hot cleaning system. Error status, output (e.g. in liters/hour), temperature, conductivity and volume can also be reported by the reverse osmosis. The concentrate mixer and the dialysis machines can also report volume requirements (“Req. volume”), for example water from the reverse osmosis, and temperature requirements (“Req. temperature”), for example the temperature of the water coming from the reverse osmosis.

The central control system can then report the operating mode, operating phase, error status, output, temperature, and volume to the components, for example. In addition, the required heat output and target temperature can be reported to the hot cleaning and/or volume requirement (“Req. volume”) and temperature requirement (“Req. temperature”) to the reverse osmosis.

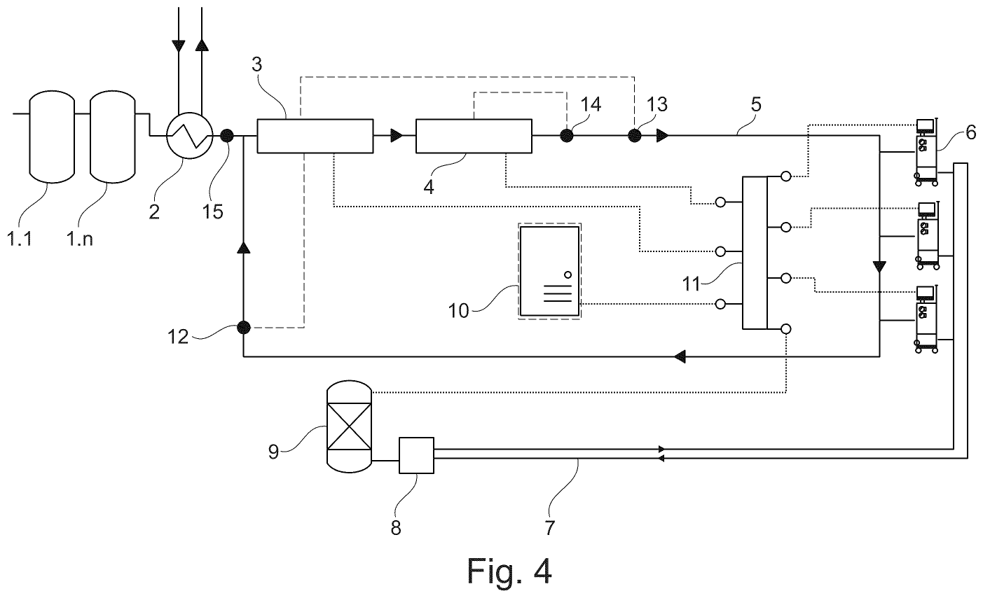

FIG. 4 shows a schematic representation of the hydraulic and information technology connections in an exemplary dialysis center comprising an exemplary dialysis system according to the present disclosure. In the figure, the dialysis system comprises the following exemplary components:
1.1 Pre-treatment unit 1
1.n Pre-treatment unit n
2 Heat exchanger
3 Reverse osmosis
4 Hot cleaning
5 Permeate ring line
6 Consumer/dialysis machine
7 Concentrate ring line
8 Concentrate distribution system
9 Concentrate mixer
10 Control device
11 Network
12 Flow meter outlet permeate ring line
13 Flow meter inlet permeate ring line
14 Temperature sensor for hot cleaning
15 Temperature sensor outlet heat exchanger

None of the above-mentioned elements, with the exception of the control device and any suitable data connections, is mandatory. Subgroups of the above elements can be suitably combined with each other. In particular, the dialysis system can be configured without a concentrate mixer 9. The dialysis system can also be configured without upstream connection of the heat exchanger and without the temperature sensor outlet heat exchanger 15.

In the following, an exemplary method according to the present disclosure is described. In describing the method, reference is made purely by way of example to the dialysis system shown in FIG. 4. However, the method can also be carried out using other suitable systems. According to the method, all components (medical devices) connected to a control device 10 via a network 11 can register their requirements, for example volume, temperature, operating mode and/or operating phase, centrally with the control device.

After determining the resource requirements, for example permeate volume requirements, temperature requirements, and/or time requirements, of all connected components/medical devices, the control device can perform leakage detection according to the present disclosure.

For example, leakage monitoring can be made possible by balancing the volume flows at the inlet 13 and outlet 12 of the permeate ring line 5 based on the registered requirements of the connected consumers.

For example, the balancing can include determining which volume flow is expected at the outlet with a certain volume flow at the inlet if the actual requirements of the connected consumers correspond to the registered requirements. If the actual volume flow at the outlet then deviates from the expected volume flow by more than a specified value, the control device can determine that there is a leakage.

Although the present disclosure is illustrated and described in detail in the figures and the foregoing description, these figures and descriptions are to be considered exemplary and not limiting. The present disclosure is not limited to the embodiments shown. In view of the foregoing description and figures, it will be apparent to those skilled in the art that various modifications can 10 be made within the scope of the present disclosure.

1. A method of operating a dialysis system comprising a plurality of hydraulically connected components and a control device, the method comprising the steps of:
receiving, by the control device, component operating data from the components, the component operating data comprising at least demand data for an upcoming task to be performed by the respective component;
determining an expected resource consumption of the component based on the demand data;
monitoring, by the control device, of an actual resource consumption of the components;
determining, by the control device, a deviation of the actual resource consumption from the expected resource consumption; and
detecting, by the control device, a leakage in the dialysis system if the deviation is outside a predetermined range.

2. The method according to claim 1, wherein the expected resource consumption is represented by one or more demand parameters and the actual resource consumption is represented by one or more consumption parameters, wherein in each case a demand parameter and a consumption parameter form a parameter pair and the deviation is determined for at least one of the parameter pairs.

3. The method according to claim 2, wherein a deviation of the actual resource consumption from the expected resource consumption is determined for several parameter pairs and a leakage is detected if at least a predetermined number of the deviations lies outside a predetermined range for the parameter pair.

4. The method according to claim 2, wherein a deviation of the actual resource consumption from the expected resource consumption is determined for several parameter pairs and a leakage is detected if at least one of the deviations is outside a predetermined range for the parameter pair.

5. The method according to claim 1, wherein the components comprise one or more consumers and wherein the monitoring of actual resource consumption comprises the control device receiving, for each of the consumers, data indicating an actual fluid withdrawal quantity.

6. The method according to claim 1, wherein the monitoring of the actual resource consumption comprises a balancing of an inflowing fluid volume of fluid flowing into a ring system and an outflowing fluid volume of fluid flowing out of the ring system.

7. The method according to claim 6, wherein the method comprises that the control device receives measurement data from one or more components of the dialysis system on the inflowing fluid volume and on the outflowing fluid volume and performs the balancing based thereon.

8. The method according to claim 1, wherein monitoring of the actual resource consumption comprises monitoring of a development over time of the actual resource consumption, and wherein determining a deviation comprises determining a deviation of the development over time of the actual resource consumption from the development over time of the expected resource consumption.

9. The method according to claim 1, wherein the method comprises receiving, by the control device, data from at least one external information system and detecting the leakage taking into account the data received from the external information system.

10. The method according to claim 1, comprising an exchange of data, initiated by the control device, between at least two of the components indirectly via the control device.

11. The method according to claim 1, wherein at least some of the components are only hydraulically connected to one another.

12. The method according to claim 1, wherein the components comprise at least one reverse osmosis device and/or at least one concentrate mixing system and/or at least one hot cleaning system and/or at least one dialysis machine.

13. A dialysis system configured to carry out the method according to claim 1, the dialysis system comprising:
a plurality of hydraulically connected components; and
a control device connected to the hydraulically connected components by a data connection.

14. A computer program product comprising instructions that cause a dialysis system to perform the steps of claim 1.

15. A computer-readable medium on which the computer program according to claim 14 is stored.
